SEQUENCE LISTING (9 sequences)

SEQ ID NO: 1 - DNA, 35 bp, Unknown
  5′-NotI-(dT)20V for first-strand cDNA synthesis
aaccctgcggccgcttttttttttttttttttttv

SEQ ID NO: 2 - DNA, 25 bp, Unknown
  oligonucleotide sequence tag template containing the terminal 3′ N as a 2′-O-Methylated nucleotide and a 3′ terminus with a C3 propyl spacer
gacgaagacagtagacannnnnnnm

SEQ ID NO: 3 - DNA, 43 bp, Unknown
  second oligonucleotide template with 5′ proximal T7 promoter sequence
aattctaatacgactcactatagggagacgaagacagtagaca

SEQ ID NO: 4 - DNA, 31 bp, Unknown
  primer with 5′ proximal AscI site and sequence tag complement to tag contained in first DNA templates (first primer)
ttggcgcgccttgggagacgaagacagtaga

SEQ ID NO: 5 - DNA, 24 bp, Unknown
  gene specific reverse primer for GAPDH (second primer)
catgtgggccatgaggtccaccac

SEQ ID NO: 6 - DNA, 32 bp, Unknown
  gene specific reverse primer for beta-and gamma-actin (second primer)
cgtcatactcctgcttgctgatccacatctgc

SEQ ID NO: 7 - DNA, 36 bp, Unknown
  gene specific reverse primer for beta-and gamma-actin with 5′ proximal NotI site for cloning
aaccctgcggccgccacatctgctggaaggtggaca

SEQ ID NO: 8 - DNA, 18 bp, Unknown
  Primer sequence in plasmid vector pCATRMAN
aatcactggacgcgtggc

SEQ ID NO: 9 - DNA, 19 bp, Unknown
  M13 reverse primer
ggaaacagctatgaccatg

(19) United States
(12) Patent Application Publication
(10) Pub. No.: US 2005/0153333 A1
(43) Pub. Date: Jul. 14, 2005

(54) Selective terminal tagging of nucleic acids

(76) Inventor: Roy Rabindranauth Sooknanan, Beaconsfield (CA)

Correspondence Address: TORYS LLP, 79 WELLINGTON ST. WEST, SUITE 3000, TORONTO, ON, M5K 1N2, CA

(21) Appl. No.: 11/000,958

(22) Filed: Dec. 2, 2004

Related U.S. Application Data
(60) Provisional application No. 60/526,074, filed on Dec. 2, 2003.

(51) Int. Cl. C12Q 1/68; C12P 19/34

(52) U.S. Cl. 435/6; 435/91.2

(57) ABSTRACT

A method is provided for adding a terminal sequence tag to nucleic acid molecules for use in RNA or DNA amplification. The method involves contacting with a mixture of oligonucleotides, each having a sequence tag template, a random sequence and a blocked 3′ terminus, under conditions such that, the random sequence anneals with the nucleic acid molecules and the nucleic acid molecules are extended using the sequence tag template as template. For synthesis of RNA from DNA molecules having terminal sequence tags, the method includes forming DNA templates having a double stranded promoter sequence and synthesizing RNA from the DNA templates. For amplification of sequences from DNA molecules having terminal sequence tags, the method includes forming DNA templates by extension of one primer having a sequence that is complementary to the terminal sequence tag and another primer having a sequence that is derived form one of the DNA molecules.

### PRIORITY CLAIM

The present application claims priority from U.S. Provisional Application No. 60/526,074 filed Dec. 2, 2003, the contents of which are incorporated herein.

### FIELD OF THE INVENTION

This invention relates to a method for adding a terminal sequence tag to nucleic acid molecules and uses thereof for RNA transcription or DNA amplification.

### BACKGROUND OF THE INVENTION

One of the more persistent objectives in molecular biology has been determining the nucleic acid sequence and relative abundance of individual species in heterogeneous mRNA populations. Methods for determining mRNA sequences typically involve analyzing the DNA sequence of single clones of a cDNA library, which are derived by enzymatic production of double-stranded cDNA from the mRNA. Methods for determining the relative abundance of mRNA species typically involve quantifying the hybridization of a defined nucleic acid sequence to a complementary sequence in the mRNA population. Analysis of samples containing a relatively low quantity of mRNA generally involves amplification prior to the application of methods for determining the sequence or relative abundance of particular mRNA species. Amplification methods that proceed with linear kinetics during the course of the amplification reaction are less likely to introduce bias in the relative levels of different mRNAs than those that proceed with exponential kinetics (Shannon, U.S. Pat. No. 6,132,997).

In Van Gelder et al., U.S. Pat. No. 5,545,522, a process is described for amplifying a target nucleic acid sequence using a single primer-promoter, an oligonucleotide that has a sequence complementary to an RNA polymerase promoter linked to a sequence complementary to the target nucleic acid sequence. In an embodiment of this process, poly(A)+ mRNA is the target nucleic acid, with a primer-promoter having a 3′-terminal oligo(dT) sequence, for the amplification of “antisense RNA”, RNA transcripts that are complementary to the original mRNA. In this embodiment, cDNA is synthesized from the mRNA by extension of the annealed primer-promoter using reverse transcriptase; the RNA strand of the resulting mRNA:cDNA hybrid is partially hydrolyzed using RNase H; a second strand of DNA is synthesized from the cDNA by extension of the annealed mRNA fragments using DNA polymerase I (Gubler et al. (1983) Gene 25: 263-269); and multiple copies of antisense RNA are synthesized from the second strand of DNA using an RNA polymerase. One problem with this method is that the 5′ ends of the mRNA, which become used as primers for second strand DNA synthesis, cannot be amplified. For 5′-terminal mRNA sequences to be included in an amplified product, an arbitrary sequence, a “sequence tag”, needs to be added to either the 5′ ends of the mRNA or the 3′ ends of the cDNA. This sequence tag provides a terminal priming site needed for amplification of the cDNA that was synthesized from the initial priming site, typically the 3′-terminal poly(A) of mRNA. Three general methods for providing a terminal priming site on mRNA or cDNA for the purposes of nucleic acid amplification are described below. Other methods based upon adding terminal polymer or oligomer tracts composed of the same nucleotide using enzymes such as terminal transfer or polyadenylate polymerase, “tailing methods”, are more applicable for cloning rather than amplifying nucleic acid molecules, and are thus not included.

In Kato et al., U.S. Pat. No. 5,597,713, a process is described for adding an arbitrary sequence to the 5′ ends of mRNA. In this process, mRNA is pretreated using a phosphatase to remove any terminal phosphates, the 5′-terminal cap is removed from the mRNA using a pyrophosphatase, and an oligonucleotide, having an arbitrary sequence composed of DNA and/or RNA, is added to the resulting 5′-terminal phosphate of the mRNA using T4 RNA ligase. In an embodiment of this process, cDNA having a 3′-terminal arbitrary sequence is synthesized from the ligated mRNA products by extension of an annealed oligo(dT) primer using reverse transcriptase. Since this process requires the performance of two hydrolytic steps on the mRNA, any contaminating hydrolytic activities in the enzymes and the alkaline reaction conditions can cause the loss of intact mRNA. In addition, T4 RNA ligase is less efficient with longer nucleic acid substrates.

In Dumas Milne Edwards et al. 1991 (Nucleic Acids Res. 19, 5227-5232) a process is described for amplifying 5′-terminal sequences of mRNA whereby an arbitrary sequence is added to the 3′ ends of cDNA. In this process, cDNA is synthesized from mRNA by extension of an annealed primer having a 3′-terminal oligo(dT) linked to a 41-nt arbitrary sequence using reverse transcriptase. After removing the mRNA from the resulting hybrid, an oligodeoxyribonucleotide, having a 44-nt arbitrary sequence, a 5′-terminal phosphate and a blocked 3′ end, is added to the 3′ ends of the cDNA using T4 RNA ligase. The ligated cDNA products, each with a different arbitrary sequence at each end, are amplified using PCR with primers derived from the 5′-terminal half of each arbitrary sequence. The resulting amplified products are purified and amplified using a second PCR this time with nested primers derived from the 3′-terminal half of each arbitrary sequence. For this process to work the optimum reaction conditions needed to be modified so that cDNA can be used as acceptor by T4 RNA ligase, resulting in the inefficient production of ligated cDNA as evidenced by the extensive exponential amplification that is required for their detection.

In Chenchik et al., U.S. Pat. No. 5,962,272, a process is described for the synthesis and cloning of cDNA corresponding to the 5′ ends of mRNA using a template-switching oligonucleotide that hybridizes to the 5′-terminal CAP of mRNA. The method comprises contacting RNA with a cDNA synthesis primer which can anneal to RNA, a suitable enzyme which possesses reverse transcriptase activity, and a template switching oligonucleotide under conditions sufficient to permit the template-dependent extension of the primer to generate an mRNA:cDNA hybrid. The template switching oligonucleotide hybridizes to the CAP site at the 5′ end of the RNA molecule and serves as a short, extended template for CAP-dependent extension of the 3′-end of the single stranded cDNA that is complementary to the template switching oligonucleotide. The resulting full-length single stranded cDNA includes the complete 5′-end of the RNA molecule as well as the sequence complementary to the template switching oligonucleotide, which can then serve as a universal priming site in subsequent amplification of the cDNA. The template switching oligonucleotide hybridizes to the CAP site at the 5′ end of the mRNA and forms basepair(s) with at least one nucleotide at the 3′ end of the cDNA of an mRNA-cDNA intermediate. Since this process is based upon the specific interaction with the CAP of an mRNA and the 3′ end of a cDNA in an mRNA-cDNA intermediate, it is unlikely to be applicable for adding terminal sequence tags to nucleic acid molecules that are single-stranded or are without a CAP structure.

The above is a cursory sampling of the methods that have been developed for the amplification of nucleic acid molecules. The person of skill in the art will be familiar with many of them and will also be familiar with their shortcomings. Some examples of the shortcomings include the sequence bias of exponential amplification and the inefficiency of single-stranded ligation; the narrow applicability to a few forms of RNA and DNA; and the requirement of a 5′-terminal CAP or an mRNA-cDNA intermediate. Notwithstanding the wide use of these amplification processes, a need exists for improvements. The research that is ongoing in this art is indicative of the search for a substantially universal method that can be broadly applied to unknown sequences in samples containing whole extractions of nucleic acids. Thus there is a need for a process that is capable of sensitive amplification of sequences from the entire mRNA, particularly from the 5′ ends.

### SUMMARY OF THE INVENTION

It is an object of the present invention to provide novel methods and kits for adding a terminal sequence tag to nucleic acid molecules and uses thereof in RNA transcription or DNA amplification, which obviates or mitigates at least one of the disadvantages of the prior art.

An aspect of the invention provides a method for adding a terminal sequence tag to nucleic acid molecules that comprises contacting the nucleic acid molecules with a mixture of oligonucleotides, each having a sequence tag template, a random sequence and a blocked 3′ terminus, under conditions such that, the random sequence anneals with the nucleic acid molecules and the nucleic acid molecules are extended using the sequence tag template as template. In particular implementations of this aspect, the nucleic molecules can be RNA or DNA. In a particular implementation of this aspect, a terminal sequence tag can be added to DNA molecules by contacting with a mixture of oligonucleotides, each having a sequence tag template, a random sequence and a blocked 3′ terminus, under conditions such that, the random sequence anneals with the DNA molecules and the DNA molecules are extended using the sequence tag template as template. In a particular implementation of this aspect, DNA molecules can be formed by contacting a mixture containing mRNA with a primer having a terminal sequence complementary to the mRNA, under conditions such that, the terminal sequence of the primer anneals with the mRNA and is extended using the mRNA as template.

Another aspect of the invention provides a method for synthesizing RNA from DNA molecules. This method comprises forming first DNA templates by adding a terminal sequence tag to the DNA molecules; forming first DNA templates having a double-stranded promoter sequence; forming second DNA templates having a double-stranded promoter and synthesizing RNA from the first or second DNA templates having a double-stranded promoter sequence. In a particular implementation of this aspect of the invention, the first DNA templates having a double-stranded promoter sequence can be formed by contacting the first DNA molecules with oligonucleotides containing the sequence tag template, a promoter template, a random sequence and a blocked 3′ terminus, under conditions such that, the random sequence anneals with the DNA molecules and the DNA molecules are extended using the sequence tag and promoter templates as template. In a particular implementation of this aspect, the second DNA templates having a double-stranded promoter sequence can be formed by contacting the first DNA templates without a promoter with a second oligonucleotide containing the sequence tag complement to the tag sequence contained in the first DNA templates and a promoter sequence template, under conditions such that, the first DNA templates anneal with the sequence tag complement of the second oligonucleotide and are extended using the promoter sequence template as template. In a particular implementation of this aspect, the second oligonucleotide can contain a blocked 3′ terminus. In a particular implementation of this aspect, a terminal sequence tag can be added to DNA molecules by contacting with a mixture of oligonucleotides, each having a sequence tag template, a random sequence and a blocked 3′ terminus, under conditions such that, the random sequence anneals with the DNA molecules and the DNA molecules are extended using the sequence tag template as template. In a particular implementation of this aspect, DNA molecules can be formed by contacting a mixture containing mRNA with a primer having a terminal sequence complementary to the mRNA, under conditions such that, the terminal sequence of the primer anneals with the mRNA and is extended using the mRNA as template.

Another aspect of the invention provides a method for synthesizing first RNA templates having a double-stranded promoter sequence comprising contacting the RNA molecules with oligonucleotides containing the sequence tag template, a promoter template, a random sequence and a blocked 3′ terminus, under conditions such that, the random sequence anneals with the RNA molecules and the RNA molecules are extended using the sequence tag and promoter templates as template. In a particular implementation of this aspect, the second RNA templates having a double-stranded promoter sequence can be formed by contacting the first RNA templates without a promoter with a second oligonucleotide containing the sequence tag complement to the tag sequence contained in the first RNA templates and a promoter sequence template, under conditions such that, the first RNA templates anneal with the sequence tag complement of the second oligonucleotide and are extended using the promoter sequence template as template. In a particular implementation of this aspect, the second oligonucleotide can contain a blocked 3′ terminus. In a particular implementation of this aspect, a terminal sequence tag can be added to DNA molecules by contacting with a mixture of oligonucleotides, each having a sequence tag template, a random sequence and a blocked 3′ terminus, under conditions such that, the random sequence anneals with the DNA molecules and the DNA molecules are extended using the sequence tag template as template. In a particular implementation of this aspect, DNA molecules can be formed by contacting a mixture containing mRNA with a primer having a terminal sequence complementary to the mRNA, under conditions such that, the terminal sequence of the primer anneals with the mRNA and is extended using the mRNA as template.

Another aspect of the invention provides a method for amplifying terminal sequences of DNA molecules comprising: forming first DNA templates by adding a terminal sequence tag to the DNA molecules; forming double-stranded DNA templates by extending a first primer; and amplifying the DNA templates by extending the first primer and a second primer. According to this embodiment the double-stranded DNA templates can be formed by contacting the first DNA templates with a first primer having a sequence complementary to the sequence tag, under conditions such that, the sequence of the primer anneals with the sequence tag of the first DNA templates and is extended. In a particular implementation of this aspect, the DNA templates can be amplified by contacting with the first primer and a second primer containing a sequence complementary to a sequence from the complementary DNA strand to the first DNA templates, under conditions such that the primers anneal to complementary templates and are extended. In a particular implementation of this aspect, a terminal sequence tag can be added to DNA molecules by contacting with a mixture of oligonucleotides, each having a sequence tag template, a random sequence and a blocked 3′ terminus, under conditions such that, the random sequence anneals with the DNA molecules and the DNA molecules are extended using the sequence tag template as template. In a particular implementation of this aspect, DNA molecules can be formed by contacting a mixture containing mRNA with a primer having a terminal sequence complementary to the mRNA, under conditions such that, the terminal sequence of the primer anneals with the mRNA and is extended using the mRNA as template.

### BRIEF DESCRIPTION OF THE FIGURES

The present invention will now be described, by way of example only, with reference to certain embodiments shown in the attached Figures in which:

FIG. 1 shows a schematic illustration of the synthesis of cDNA molecules from mRNA molecules

FIG. 2 shows a schematic illustration of the synthesis of first DNA templates comprising the oligonucleotide sequence tag from cDNA molecules

FIG. 3 shows a schematic illustration of the synthesis of first RNA templates comprising the oligonucleotide sequence tag from RNA molecules

FIG. 4 shows a schematic illustration of the synthesis of second DNA templates containing a promoter sequence fron the first DNA templates

FIG. 5 shows agarose gel electrophoretic analysis of the products of transcription reactions from cDNA prepared with or without a terminal sequence tag, as detected by ethidium bromide staining (A) or by blot hybridization with ³²P labeled cDNA probes GAPDH (B) and β-actin (C)

FIG. 6 shows agarose gel electrophoretic analysis of products from PCR using cDNA prepared with or without a terminal sequence tag and a common forward primer (first primer) in combination with gene specific reverse primers (second primers) for GAPDH and actin, as detected by ethidium bromide staining (A) or by blot hybridization with ³²P labeled cDNA probes GAPDH (B) and β-actin (C)

FIG. 7 shows agarose gel electrophoretic analysis of the products of transcription reactions from cDNA prepared with a terminal sequence tag, as detected by ethidium bromide staining (A) or by blot hybridization with ³²P labeled cDNA probes to Cathepsin K (B)

FIG. 8 shows a plot of the hybridization signal of the probe to Cathepsin K, quantified by scintillation counting of bands excised from the hybridized blot shown in FIG. 7B, versus the fraction of osteoclast RNA in the RNA mixture.

### DETAILED DESCRIPTION

The present invention relates to methods and kits for adding a terminal sequence tag to nucleic acid molecules and uses thereof in RNA or DNA amplification. Nucleic acid molecules with a terminal sequence tag and amplified RNA and DNA derived therefrom can have a variety of utilities including the generation of hybridization probes, the construction of cDNA libraries, and the analysis of terminal nucleic acid sequences.

According to a present embodiment of the invention, a terminal sequence tag is added to nucleic acid molecules. As used herein, the nucleic molecules can be DNA or RNA. DNA molecules can be complementary DNA (cDNA) formed by contacting a mixture containing mRNA with a primer having a terminal sequence complementary to the mRNA, under conditions such that, the terminal sequence of the primer anneals with the mRNA and is extended using the mRNA as template. The cDNA can be prepared from total RNA or purified mRNA, using oligo(dT) as a primer and reverse transcriptase for extending the primer. The RNA can be removed from the cDNA by using chemical, enzymatic, mechanical or thermal methods. DNA molecules can be formed by making double-stranded DNA single-stranded by using chemical, enzymatic, mechanical or thermal methods. RNA can be any ribonucleic acid molecule or library of ribonucleic acid molecules containing a 3′-OH group. Double-stranded RNA can be made single-stranded by using chemical, enzymatic, mechanical or thermal methods.

Nucleic acid molecules can be contacted with a mixture of oligonucleotides, each having a 5′ terminal sequence tag template, a random sequence and a blocked 3′ terminus. The 5′ terminal sequence tag template is an arbitrary sequence that can be any combination of purines and pyrimidines, including but not limited to G, A, T or C (natural or modified) arranged to form a sequence of any desired length. The sequence tag template should be of a particular length and base composition to provide a template for accurate extension of the nucleic acid molecules. The sequence tag template could be deoxy- and/or ribonucleotides once it provides a template for the enzymatic extension of the nucleic acid molecules. The sequence tag template should not contain sequences that are commonly found among the particular population of nucleic acid molecules. The sequence tag template should be substantially free of symmetry elements, such as direct and inverse repeats, and it should provide a template for extension of the nucleic molecules in forming a 3′-terminal sequence tag. The complement to this sequence should also provide a sequence tag that can be used as a site for hybridizing and extending an oligonucleotide primer or for hybridizing an oligonucleotide template, which can be used for extension of the tagged nucleic acid molecules. The 3′-proximal random sequence can be any number of nucleotides in length but preferably between about 4 and about 9 and comprising an equal representation of G, A, T and C at each of the different positions. Wobble bases such as inosine (I) can also be used instead of the standard bases at any of the positions. In addition, one or more of the nucleotides contained in the 3′ proximal random sequence can be chemically modified for example, 2′-O methylated nucleotides, phosphorothioates or any such chemical modifications that render the nucleotide(s) inert to nucleases. The 3′ terminus of the oligonucleotides is chemically blocked with, for example, C3 propyl spacer, amine group (NH₂), phosphate or any other chemical modifications that render the oligonucleotide mixture inert as a primer for primer extension using either a DNA- or RNA-directed DNA polymerase.

Reaction conditions are applied such that, the random sequences of the oligonucleotides can anneal with the nucleic acid molecules and the nucleic acid molecules can be extended using as template the sequence tag template of the oligonucleotides. The oligonucleotides and nucleic acid molecules are allowed to anneal by heating a mixture of these two components at an elevated temperature (greater than about 37° C.) for a period of time and then incubating at a temperature that is desirable for enzymatic extension of the nucleic acid molecules, typically about 37° C. The nucleic acid molecules are extended by using a DNA polymerase, which can be any enzyme capable of synthesizing DNA by extending a DNA or RNA primer using a DNA or RNA template. The DNA polymerase should not have exonuclease activities, either 3′ to 5′ or 5′ to 3′, and preparations containing the DNA polymerase should be substantially free of agents capable of nucleic acid hydrolysis. Examples of DNA polymerase that can be used include [Klenow exo⁻ DNA polymerase, Bst DNA polymerase, AMV and M-MLV reverse transcriptases.

The DNA polymerase reaction comprises the desirable concentrations of cofactors and deoxynucleoside triphosphates for DNA synthesis using the particular DNA polymerase and is performed under the conditions of pH, ionic strength and temperature that are desirable for the enzyme that is used. Such reaction conditions are known to those skilled in the art. The reaction is performed for a sufficient period of time to allow extension of the nucleic acid molecules using the oligonucleotides as template. The reaction can be terminated using any chemical, enzymatic, mechanical or thermal methods, and the extended nucleic acid molecules can be purified from the unused oligonucleotides using size exclusion or any other suitable separation method known in the art. The resulting nucleic acid molecules have a 3′-terminal sequence tag that is complementary to the sequence tag template contained in the oligonucleotide mixture.

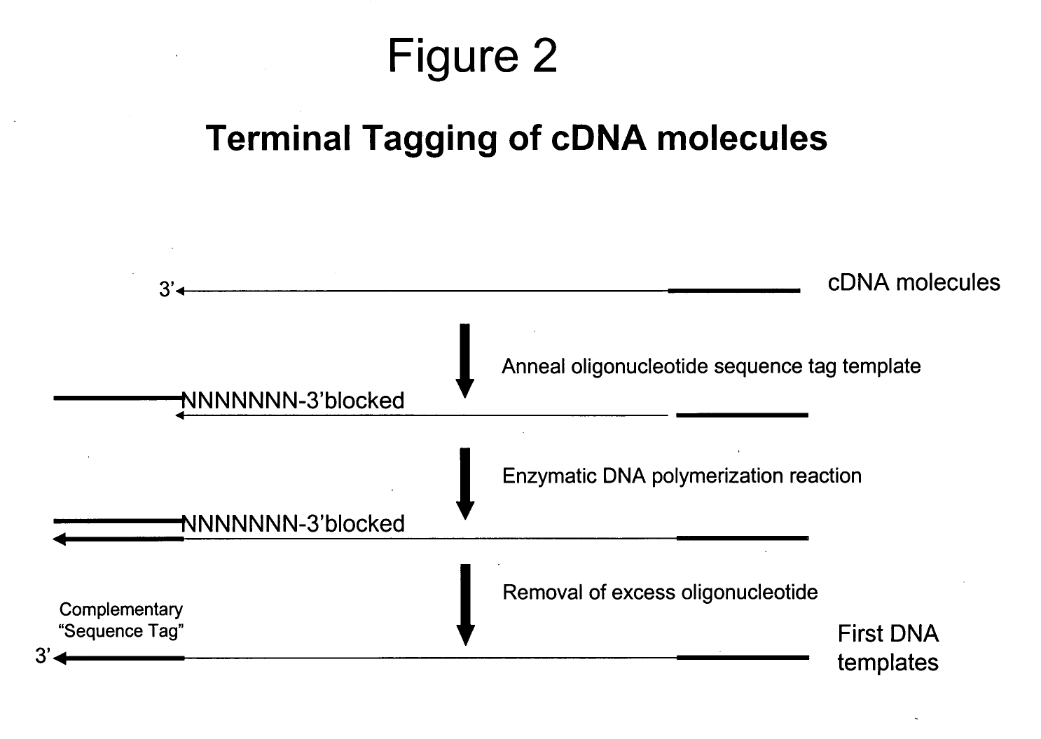

According to a present embodiment, the nucleic acid molecules can be composed of DNA, wherein the resulting “first DNA templates” have a 3′-terminal sequence tag that is complementary to the sequence tag template contained in the oligonucleotide mixture (see schematic of FIG. 2 for illustration).

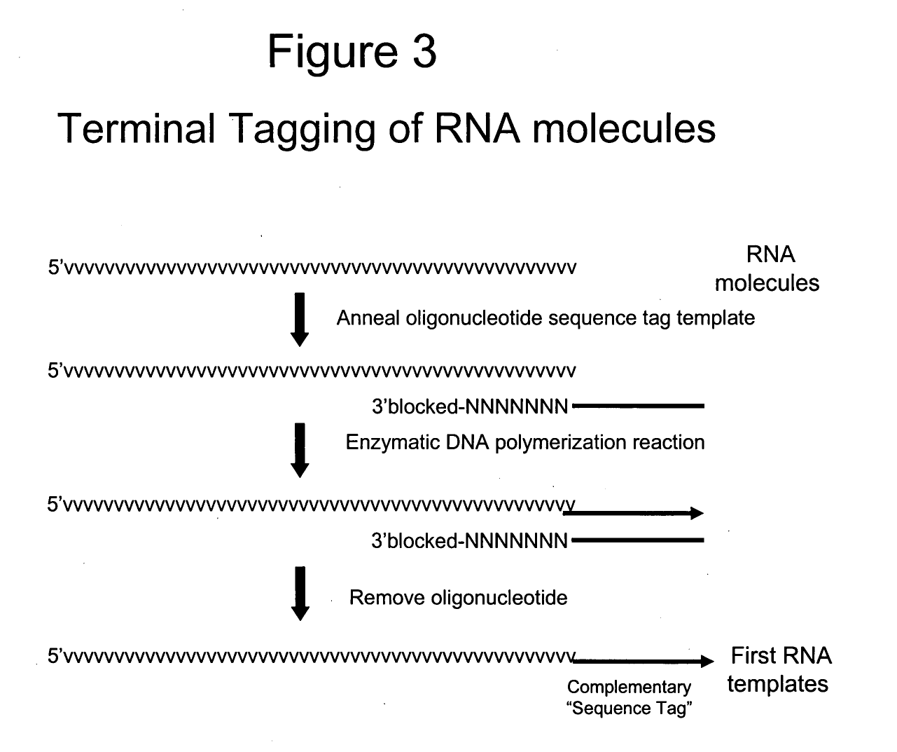

According to a present embodiment, the nucleic acid molecules can be composed of RNA, wherein the resulting “first RNA templates” have a 3′-terminal sequence tag that is complementary to the sequence tag template contained in the oligonucleotide mixture. The first RNA templates formed comprise a composite of deoxy- and ribonucleotides (see schematic of FIG. 3 for illustration).

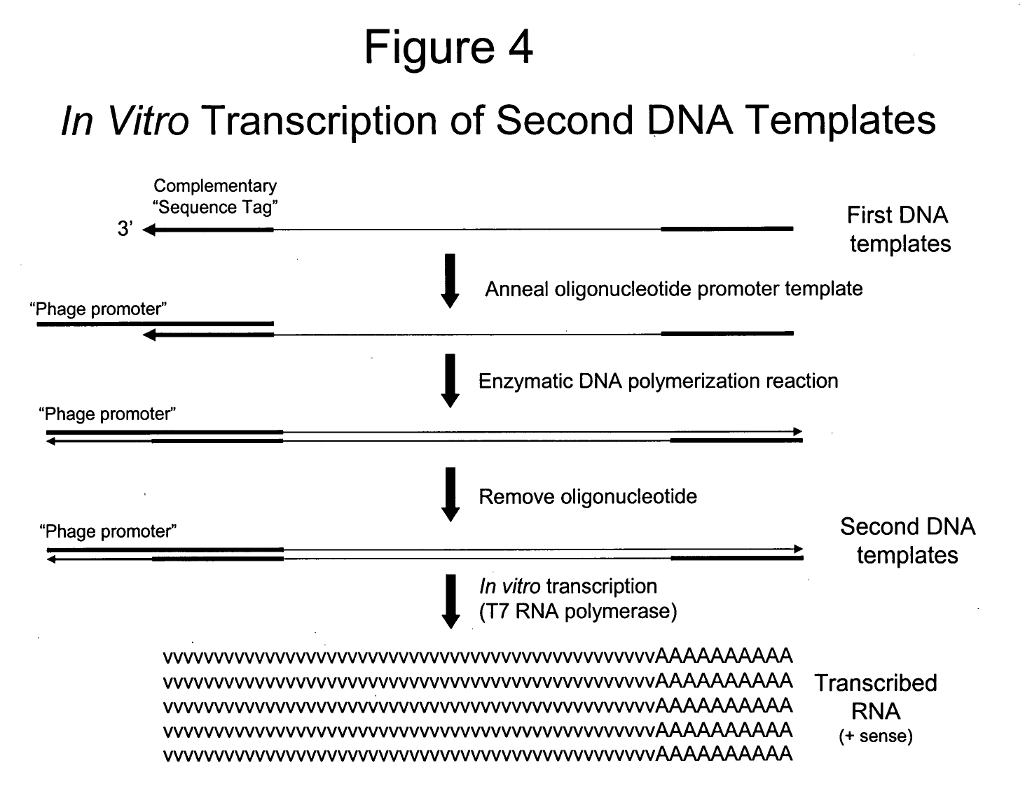

According to a present embodiment, RNA is synthesized from DNA molecules by forming first DNA templates, forming first DNA templates having a double-stranded promoter sequence, forming second DNA templates having a double-stranded promoter sequence (see schematic of FIG. 4 for illustration) and synthesizing RNA from the first or second DNA templates having a double-stranded promoter sequence.

According to a present embodiment, second DNA templates having a double-stranded promoter sequence can be formed by contacting the first DNA templates without a promoter with a second oligonucleotide, containing a sequence tag complement and a promoter template. The sequence tag complement is a sequence near the 3′ end of the second oligonucleotide that is complementary to the 3′-terminal sequence tag of the first DNA templates. The sequence tag complement is of a particular length and base composition to allow specific and efficient annealing to the sequence tag of the first DNA template under conditions, presently preferred to be those of an enzymatic DNA polymerization reaction. The second oligonucleotide should also have a 3′-terminal sequence that reduces annealing to itself or another primer in the reaction such that a primer would be extended using itself or another primer as template in a DNA or RNA amplification reaction, hence producing what is described in the art as “primer-dimers”. The promoter template is a sequence near the 5′ end of the second oligonucleotide that contains the plus (+) sense sequence of a promoter and its transcription initiation site. The promoter template is of a particular length and base composition to allow specific and desirable synthesis of double-stranded promoters by extension of the first DNA templates under the conditions of an enzymatic DNA polymerization reaction. The resulting double-stranded promoter contains sufficient information to allow specific and desirable binding of an RNA polymerase and initiation of transcription at the desired site. In a presently preferred embodiment, the promoter and initiation sequences are specific for the RNA polymerase from the bacteriophage T7. In addition promoters for other RNA polymerases, such as phage T3 or Salmonella phage sp6, can be used. Furthermore, the second oligonucleotide can have a blocked 3′ terminus, which prevents it from functioning as a primer for primer extension using the first DNA templates as template to form complete double-stranded DNA templates. Furthermore, the second oligonucleotide can contain at its 5′ terminus or embedded, sequence(s) corresponding to selected restriction endonucleases. First DNA templates will be extended once annealed to the second oligonucleotide at the complementary sequence tag region to form double-stranded “activated” restriction endonuclease site(s). It will occur to those of skill in the art that other suitable promoter and initiation sequences can be used to achieve desirable levels of transcription.

According to a present embodiment, second DNA templates comprising fully double-stranded molecules minus a promoter sequence can be formed by contacting first DNA templates with a second oligonucleotide containing a sequence tag complement minus the promoter template of a particular length and base composition to allow specific and efficient synthesis of a double-stranded DNA by extension of the second oligonucleotide primer using the first DNA templates as template in an enzymatic DNA polymerization reaction. The second oligonucleotide should also have a 3′-terminal sequence that reduces annealing to itself or another primer in the reaction such that a primer would be extended using itself or another primer as template in a DNA amplification reaction, hence producing what is described in the art as “primer-dimers”. Furthermore, the second oligonucleotide can contain at its 5′ terminus or embedded, sequence(s) corresponding to selected restriction endonucleases. First DNA templates will be extended once annealed to the second oligonucleotide at its complementary sequence tag region to form double-stranded “activated” restriction endonuclease site(s). It is contemplated that the second oligonucleotide can be composed in part of nucleotides other than deoxyribonucleotides provided that a first primer of such composition can still function as template for DNA Reaction conditions are applied such that, the sequence tag of the first DNA template can anneal with the sequence tag complement of the second oligonucleotide and the first DNA template can be extended using the promoter sequence of the second oligonucleotide as template. The reaction comprises the first DNA templates, the second oligonucleotide, a DNA polymerase, deoxyribonucleoside triphosphates and the appropriate reaction buffer. The reaction is allowed to proceed at selected temperatures and for sufficient time to enable the terminal sequence tag of the DNA templates and its complementary sequence tag of the second oligonucleotide to anneal and the 3′-end of the DNA templates extended with the second oligonucleotide serving as the template resulting in the DNA templates having a double-stranded promoter. DNA polymerization from the 3′ end of the second oligonucleotide can also proceed concomitantly using the DNA templates as the template.

According to a present embodiment, the RNA polymerase, which is used in this invention can be any enzyme capable of recognizing the double-stranded promoter and specifically initiating RNA synthesis at the defined initiation site within close proximity to the promoter. Preparations comprising the RNA polymerase should be relatively free of contaminating agents with DNase or RNase activities. In addition the RNA polymerase should be capable of synthesizing several copies of RNA per functional copy of DNA template in a desirable period of time. In a presently preferred embodiment T7 RNA polymerase is used. In addition other suitable bacteriaphage RNA polymerases, such as phage T3 or Salmonella phage sp6, can be used. It is understood by those skilled in the art that the use of alternative RNA polymerases will involve changes to the sequence of the promoter template according to the specificity of the particular RNA polymerase.

The transcription reaction comprises the desirable concentrations of cofactors and nucleoside triphosphates for RNA synthesis using the particular RNA polymerase. The transcription reaction is performed under the conditions of pH, ionic strength and temperature that are desirable for the enzyme which is used. Such reaction conditions are known to those skilled in the art.

According to a present embodiment, RNA can be synthesized from the first or second DNA templates having a double-stranded promoter sequence by using an RNA polymerase that is specific to the particular promoter sequence. The reaction comprises the DNA templates, a RNA polymerase buffer [40 mM Tris-HCl (pH 7.9), 6 mM MgCl₂, 2 mM spermidine, 10 mM DTT] supplemented with an equimolar mixture of ATP, UTP, GTP and CTP incubated at about 37° C. for a specified period.

According to a present embodiment, terminal sequences of DNA molecules are amplified by forming first DNA templates, forming second DNA templates, and amplifying the DNA templates. Double-stranded DNA templates can be formed by contacting the first DNA templates with a first primer having a sequence complementary to the sequence tag contained in the first DNA templates, under conditions such that, the sequence of the primer anneals with the complementary sequence tag in the first DNA templates and is extended using the first DNA templates as template. The first primer can contain at its 5′-terminus or embedded, a sequence corresponding to any selected restriction endonuclease. The restriction endonuclease sequence is to aid in cloning of the amplified DNA templates. The first primer is extended using the first DNA templates as template in order to form the double-stranded DNA templates in a reaction comprising a DNA polymerase, deoxyribonucleoside triphosphates and the desirable reaction buffer at selected temperatures desirable to primer extension.

Terminal nucleic acid sequences can be amplified by contacting the first DNA templates with the first primer and its complementary strands with a second primer, containing a sequence complementary to a sequence from its complementary strands, under conditions such that the primers anneal to the complementary DNA strands and are extended. The second primer can contain a sequence that is complementary to one or more specific sequence(s) in a mixture of second DNA templates, whereby a limited number of terminal sequences in a mixture of DNA templates are amplified in vitro using, for example, technologies such as PCR, NASBA and SDA. Furthermore, the second primer can contain at its 5′-terminus a sequence corresponding to any selected restriction endonuclease in order to aid in cloning of the amplified DNA templates.

The present invention also relates to various kits that can be formed in order to perform the various methods of the present invention. Examples of kits include combinations of two or more reagents used in the methods. Specific examples of kits can include a mixture of oligonucleotides and a DNA polymerase. A kit can further include an oligonucleotide having a promoter sequence and an RNA polymerase. A kit can also further include a reverse transcriptase and an oligonucleotide complementary to mRNA molecules.

While only specific combinations of the various features of the present invention have been discussed herein, it will be apparent to those of skill in the art that desired sub-sets of the disclosed features and/or alternative combinations of these features can be utilized.

It will be apparent to those skilled in the art that various modifications and variations can be made in the apparatus and methods of the present invention without departing from the spirit or scope on the invention. Thus, it is intended that the present invention covers the modifications and variations of this invention provided they come within the scope of the appended claims and their equivalents. Additionally, the following examples are appended for the purpose of illustrating the claimed invention, and should not be construed so as to limit the scope of the claimed invention.

### EXAMPLE 1

### Attachment of an Oligonucleotide Sequence Tag to the Terminal 3′ Ends of cDNA Molecules

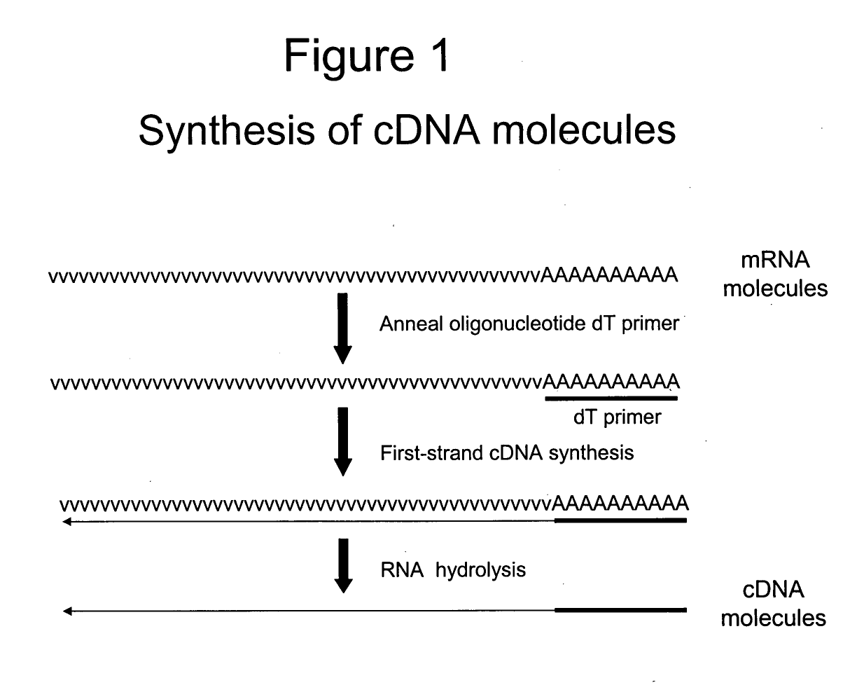

Total RNA from mouse brain (Ambion) was repurified using the RNeasy procedure (Qiagen). The mRNA population contained in 4 μg of total RNA was used for making first-strand cDNA in a standard cDNA synthesis reaction containing 7.5 μM oligo dT primer (Seq. ID. No. 1; (dT)₂₀V containing a 5′-Not I restriction endonuclease sequence in order to facilitate cloning), 10 mM Tris-HCl (pH 8.3), 50 mM KCl, 6 mM MgCl₂, 5 mM DTT, 1 mM dATP, 1 mM dGTP, 1 mM dCTP, 1 mM TTP and a reverse transcriptase in a final volume of 20 μL. The reaction was allowed to proceed for 60 minutes at the recommended incubation temperatures. The RNA templates were then removed by enzymatic digestion with RNase A and H simultaneously, and the cDNA purified and recovered in 50 μL EB buffer (Qiagen) (see schematic of FIG. 1 for illustration).

The purified first-strand cDNA molecules were then divided into 2 equal aliquots and dried. To the first aliquot, 1.5 nmol (7.5 μL) of the oligonucleotide sequence tag template (Seq. ID. No. 2; GACGMGACAGTAGACANₓ(N(2′-O-Methyl))(3′-C3 propyl spacer)) was added and to the second aliquot, 7.5 μL water. Both aliquots were incubated at 65° C. for 5 min and then at 37° C. for 10 min. Thereafter, each aliquot was adjusted to 20 μL by adding components of a DNA synthesis reaction, at final concentrations of 1 mM Tris-HCl (pH 7.5), 0.5 mM MgCl₂, 0.75 mM DTT, 33 μM dATP, 33 μM dGTP, 33 μM dCTP, 33 μM TTP and 0.5 units/μL Klenow fragment (3′ to 5′ exo⁻) (New England Biolabs). The reactions were incubated for an additional 60 minutes at 37° C. and then terminated with the addition of phenol. The first DNA templates formed in reaction 1 (see schematic of FIG. 2 for illustration) was then purified from any excess sequence tag oligonucleotide by size selection (Amersham) in a final volume of approximately 40 uL. The first-strand cDNA molecules from reaction 2 was similarly purified although it did not contain any oligonucleotide sequence tag template.

### EXAMPLE 2

### Transcription of the First DNA Templates

The DNA templates from each of the 2 reactions in Example 1 were used for priming DNA synthesis using a second oligonucleotide template containing a 5′T7 promoter sequence (italicized) and a 3′ sequence tag complement (Seq. ID. No. 3; AATTCTAATACGACTCACTATAGGGAGACGMGACAGTAGACA) to the sequence tag contained in the first DNA templates to form second DNA templates containing a T7 promoter sequence. The DNA synthesis reactions (50 uL) contained the respective DNA templates, 5 pmoles second oligonucleotide template (Seq. ID. No. 3), 40 mM Tricine-KOH (pH 8.7), 15 mM KOAc, 3.5 mM Mg(OAc)₂, 3.75 μg/mL BSA, 0.005% Tween-20, 0.005% Nonidet-P40, 200 μM dATP, 200 μM dGTP, 200 μM dCTP, 200 μM TTP and 2 μL Advantage 2 Polymerase mix (BD Biosciences). The reactions were heated at 95° C. for 1 minute 30 seconds, 50° C. for 1 minute, 55° C. for 1 minute and finally, 68° C. for 30 minutes before phenol was added to terminate the reaction. In addition to DNA synthesis primed from the first DNA templates using the second oligonucleotide template as template, DNA synthesis could as well be primed from the second oligonucleotide template using the first DNA templates as template in the same reaction (see schematic of FIG. 4 for illustration) to form completely double-stranded second DNA templates. The resulting DNA templates from both reactions were purified by size selection (Amersham) and transcribed in vitro.

Each in vitro transcription reaction (40 μL) comprised the respective DNA templates, 40 mM Tris-HCl (pH 7.9), 6 mM MgCl₂, 2 mM spermidine, 10 mM DTT, 0.5 mM ATP, 0.5 mM GTP, 0.5 mM CTP, 0.5 mM UTP and 4 μL T7 RNA polymerase (Ambion). The reactions were incubated at 37° C. for at least 2 hours, digested with DNase I at 37° C. for 30 minutes, phenol extracted and purified. An equal quantity from each transcription reaction was analyzed by agarose gel electrophoresis and Northern blot hybridization with ³²P labeled cDNA probes for GAPDH and β-actin (BD Biosciences).

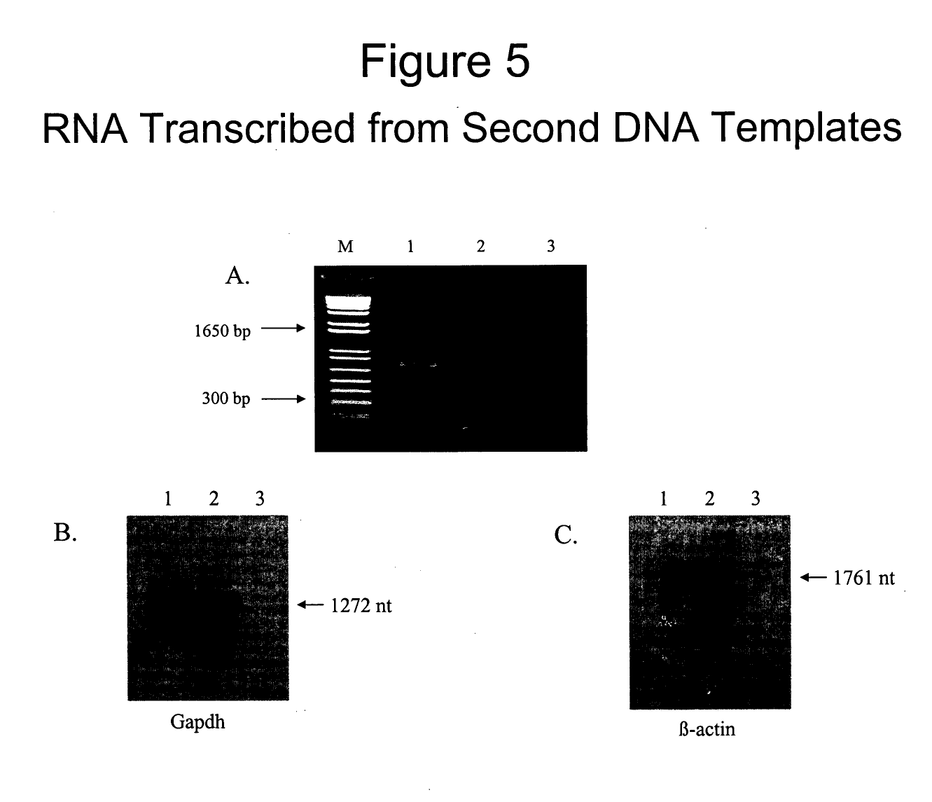

As shown in FIG. 5, at A, Lane 1 contains 200 ng of neat total RNA from mouse brain, Lane 2 contains a 4-μL aliquot of the transcription reaction from the second DNA templates containing the T7 promoter sequence and Lane 3 contains a 4-μL aliquot of the transcription reaction from DNA templates prepared without the addition of the oligonucleotide sequence tag template (cDNA molecules) (Seq. ID. No. 2). In Lane 2, RNA of various sizes (a RNA smear ranging from ˜300 bp to ˜1650 bp based on the 1 Kb Plus DNA ladder (InVitrogen)), as expected from a library of cDNA molecules, were synthesized from the second DNA templates whereas, in Lane 3, no such RNA was observed. The Northern blot analysis (FIG. 5 at B and C, Lane 2) confirms the presence of both GAPDH and β-actin sequences in the amplified RNA, and the majority of the transcribed RNA species corresponding to these two genes migrated at approximately the expected full-length molecular weight positions in comparison to the respective full-length bands (GAPDH (1272 bp; mRNA Accession # X01677) and β-actin (1761 bp; mRNA Accession # X00351) seen for the neat total RNA (FIG. 5 at B and C, Lane 1). Also, there was no hybridization signal seen for either gene when no transcribed RNA synthesized was present (FIG. 5 at B and C, Lanes 3). These results suggest that the preferred reaction for the attachment of the second oligonucleotide template containing the promoter sequence (Seq. ID. No. 2) was primarily at the 3′-ends of the first DNA templates.

FIG. 5 contains the following:
Lane 1-200 ng total RNA from mouse brain
Lane 2-4 μL transcribed RNA from second DNA templates
Lane 3-4 μL transcribed RNA from cDNA molecules

### EXAMPLE 3

### Amplification in PCR of Specific DNA Sequences Contained in a Library of First DNA Templates Using a First Primer Corresponding to the Oligonucleotide Sequence Tag and Gene Specific Second Primers

In vitro transcribed RNA (5 μg) generated in Example 2 containing the oligonucleotide sequence tag at its 5′ proximal end was reverse transcribed in a standard cDNA synthesis reaction (In Vitrogen) and the resulting first-strand cDNA was purified and reconstituted in 20 μL H₂O. Four PCR amplification reactions were assembled, each containing 40 mM Tricine-KOH (pH 8.7), 15 mM KOAc, 3.5 mM Mg(OAc)₂, 3.75 μg/mL BSA, 0.005% Tween-20, 0.005% Nonidet-P40, 200 μM dATP, 200 μM dGTP, 200 μM dCTP, 200 μM TTP and 2 μL Advantage 2 Polymerase mix in a final volume of 50 μL. To reactions 1 and 2, 20 picomoles of each of a forward primer (first primer) (Seq. ID. No. 4; TTGGCGCGCCTTGGGAGACGMGACAGTAGA), which is complementary to the sequence tag on the 3′ proximal end of the synthesized cDNA and a gene specific reverse primer (second primer) for GAPDH (Seq. ID. No. 5; CATGTGGGCCATGAGGTCCACCAC) were added. Similarly, to reactions 3 and 4, 20 picomoles of each of the same first primer (Seq. ID. No. 4) and instead, a specific reverse primer (second primer) for β- or γ-actin (Seq. ID. No. 6; CGTCATACTCCTGCTTGCTGATCCACATCTGC) were added. Additionally, to reactions 2 and 4, 2-μL aliquots of the reverse transcribed first-strand cDNA templates were added, whereas, to reactions 1 and 3, 2-μL aliquots of water were added instead. All four reactions were amplified using PCR for 25 cycles—each cycle comprising 95° C. for 1.5 minutes, 55° C. for 2 minutes and 68° C. for 3 minutes followed by a final extension at 68° C. for 30 minutes. A 5-μL aliquot from each reaction was analyzed by agarose gel electrophoresis and Southern blot hybridization with ³²P labeled cDNA probes for GAPDH and β-actin (BD Biosciences).

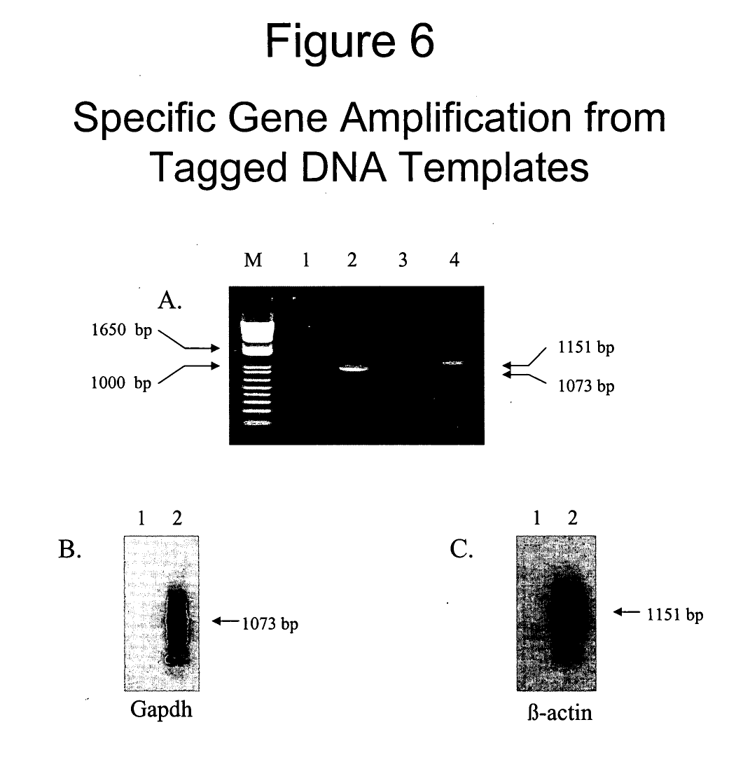

As shown in FIG. 6 at A, Lanes 1 and 3, which contained no tagged cDNA, gave no amplified products and only the primers were visible. On the other hand, Lanes 2 and 4 contained amplified products and in each case, a major product band was observed migrating at the expected molecular weight for the GAPDH (1073 bp) or β-actin (1151 bp) products respectively, which corresponded to the sequence tag present at the proximal 3′ ends of the respective full-length cDNA species.

Southern blot analysis (FIG. 6 at B and C, Lanes 2 and 4) confirms the amplified products as GAPDH and β-actin respectively. It is also possible that the β-actin probe will hybridize to γ-actin sequences, which will be amplified by these primers as well.

FIG. 6 contains the following:
Lane 1—no added template
Lane 2—2 μL aliquot of oligo-tagged first-strand cDNA as template

### EXAMPLE 4

### Verification of the Presence of the Oligonucleotide Sequence Tag at the 3′-ends of First DNA Templates

A 2-μL aliquot of the PCR-amplified materials for β-/γ-actin as generated in Example 3, reaction #4, was used as template in a secondary PCR reaction containing the first primer (Seq. ID. No. 4) and a gene specific reverse primer for β-/γ-actin (Seq. ID. No. 7; MCCCTGCGGCCGCCACATCTGCTGGAAGGTGGACA) now containing a 5′ Not I restriction endonuclease site to aid in cloning. The PCR reaction was performed as described in Example 3. The completed PCR reaction was then purified using the Qia-PCR clean-up procedure (Qiagen) and products corresponding to 50% of the purified reaction was concentrated and separated by agarose gel electrophoresis. A major product band corresponding to actin was then excised and digested with restriction endonucleases Asc I and Not I, in a 50-μL reaction comprising 20 mM Tris-acetate 9 (pH 7.9), 50 mM KOAc, 1 mM DTT, 100 μg/mL BSA and 10 units of each enzyme (NEB). The digestion reaction was incubated at 37° C. for 3 hours, purified using the Qia-PCR clean-up procedure, concentrated into a 2-μL aliquot and used in a ligation reaction. The ligation reaction comprised Asc I-Not I digested PCR amplicons (2 μL) and 20 ng plasmid vector (pCATRMAN) for cloning in E. coli, 50 mM Tris-HCl (pH 7.5), 10 mM MgCl₂, 10 mM DTT, 1 mM ATP, 25 μg/mL BSA and 400 units T4 DNA ligase (NEB). The ligation reaction was incubated at 16° C. overnight, which was followed by 65° C. for 10 minutes. To the ligation reaction, 90 μL H₂O and 1 mL butanol were added, mixed, and the precipitate collected by centrifugation and reconstituted in 4 μL H₂O. A 2-μL aliquot was then used to transform E. coli (DH10B) by electroporation (Invitrogen). After incubating the electroporated cells at 37° C. in 1 mL SOC complete media (Sambrook et al., 1990) for 1 hour, 1 μL and 10 μL aliquots were plated on YT agar plates containing 100 μg/mL ampicillin and grown at 37° C. overnight. Next, 30 colonies were picked directly into 50 μL aliquots of H₂O and 43 μL of each aliquot added to individual PCR reactions comprising 10 μL 10× reaction buffer (Qiagen), 0.2 mM dATP, 0.2 mM dGTP, 0.2 mM dCTP, 0.2 mM TTP, 20 μmoles forward primer (Seq. ID. No. 8; MTCACTGGACGCGTGGC), 20 μmoles reverse primer (Seq. ID. No. 9; GGAAACAGCTATGACCATG) and 3 units Hot start Taq DNA polymerase (Qiagen). The reactions were heated at 99° C. for 10 minutes followed by 30 cycles of 95° C. for 1.5 minutes, 55° C. for 1 minute, 72° C. for 2 minutes and a final extension at 72° C. for 15 minutes. A 5-μL aliquot of each reaction was then analyzed by agarose gel electrophoresis for the presence of PCR amplicons before proceeding to sequence analysis.

For sequence analysis, a 5-μL aliquot from 24 amplification reactions containing a PCR amplicon was used in the Big Dye Automated DNA sequencing procedure (Applied Biosystems Inc.) using Seq. ID. No. 8, as the sequencing primer. Table 1 below shows the first 80 nucleotides of 5′ terminus of the DNA sequences obtained for the γ-actin clones sequenced. It appears that each of the 24 clones contained a sequence corresponding to γ-actin rather than β-actin. More important though, in each case, the oligonucleotide sequence tag (Seq. ID. No. 2) was present at the 3′ proximal end of all cDNA fragments cloned for γ-actin (shown as bold and italicized fonts) whether or not, the cDNA fragment was synthesized to the extreme 5′-terminus of the mRNA species or terminated prematurely at various positions during the cDNA synthesis reaction. The additional 4 nucleotides (GGGA) upstream of the tag sequence represent the transcription initiation site of the T7 promoter. In general, the majority of the clones contain the tag sequence affixed at the 5′ terminus of the known full-length sequences γ-actin (from 4 bases upstream (−4) of Accession # BC023248.1 to 8 bases (+8) downstream of Accession # AK076081.1) (Table 1). However, there were some clones for γ-actin (Table 1, clones #22, #23 and #24) that were tagged at different positions more internally, which likely represented different termination positions during cDNA synthesis. These results clearly indicate that regardless of the terminal sequence at the 3′-ends of cDNA fragments, an appropriate oligonucleotide sequence tag will likely become appended following the teachings as described in Example 1.

TABLE 1

| | | | 5′ Terminus |
|---|---|---|---|
| | | | relative to |
| | | | Accession # |
| SPECIFIC | CLONE | | BC023248.1/ |
| SEQUENCE | # | Sequences (5′-80 nt) | AK076081.1 |
| γ-actin | 1-18 | GGGA [symbol] CTCCGCCGCCGGCTTACAC | −2/+8 |
| | | TGCGCTTCTTGCCGCTCCTCCGTCGCCGCCGCGTCCTTCG | |
| | 19-21 | GGGA [symbol] CACTCCGCCGCCGGCTT | −4/+6 |
| | | ACACTGCGCTTCTTGCCGCTCCTCCGTCGCCGCCGCGTC | |
| | | CTTCG | |
| | 22 | GGGA [symbol] CGGGGTCACACACACAGTG | +537/+546 |
| | | CCCATCTATGAGGGCTACGCCCTTCCCCACGCCATCTTGC | |
| | 23 | GGGA [symbol] TTCAGGCGGTGCTGTCCT | +473/+482 |
| | | TGTATGCATCTGGGCGCACCACTGGCATTGTCATGGACTCT | |
| | 24 | GGGA [symbol] AGCTAACAGAGAGAAGAT | +405/+414 |
| | | GACGCAGATAATGTTTGAAACCTTCAATACCCCAGCCATGT | |

Table 1 shows a summary of approximately the first 80 nucleotides from the 5′ ends of the γ-actin clones that were sequenced to demonstrate the presence of the oligonucleotide sequence tag (shown italicized and bolded).

### EXAMPLE 5

### Linear Transcription of Libraries of Second DNA Templates as Demonstrated by the Detection of a Specific Gene Sequence (Cathepsin K)

Total RNA from undifferentiated (precursor) and fully differentiated (osteoclast) mouse RAW 264.7 cells was extracted using a Trizol method (InVitrogen), purified further by RNeasy (Qiagen) and quantified at A_{260 nm}. The precursor and osteoclast specific total RNA samples were then mixed in the following ratios:
1. 500 ng precursor+0 ng osteoclast total RNA
2. 400 ng precursor+100 ng osteoclast total RNA
3. 250 ng precursor+250 ng osteoclast total RNA
4. 100 ng precursor+400 ng osteoclast total RNA
5. 0 ng precursor+500 ng osteoclast total RNA

First-srand cDNA was then synthesized from each RNA or RNA mixture and first DNA templates prepared using the oligonucleotide sequence tag (Seq. ID. No. 2) according to the teachings of Example 1. Each first DNA templates was subsequently annealed to a second oligonucleotide template containing a T7 promoter sequence and a oligonucleotide sequence tag complement to tag sequence contained in the first DNA templates (Seq. ID. No. 3) and an enzymatic DNA polymerization reaction for each performed as described in Example 2. The resulting second DNA templates containing a double-stranded T7 promoter for each reaction was purified and transcribed in vitro using T7 RNA polymerase as described in Example 2. An equal amount of RNA (500 ng) from each transcription reaction was analyzed by agarose gel electrophoresis and Northern blot hybridization to a ³²P labeled cDNA probe specific for mouse cathepsin K gene.

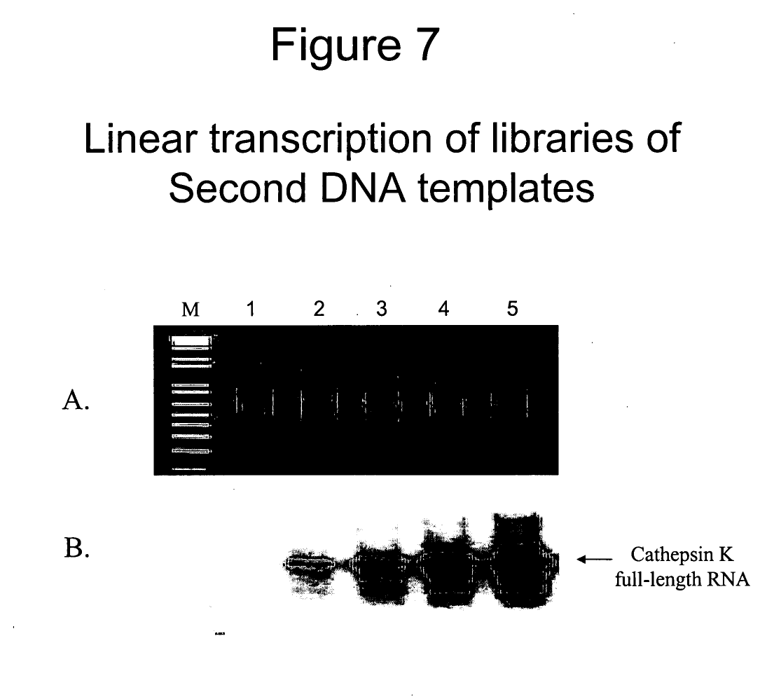

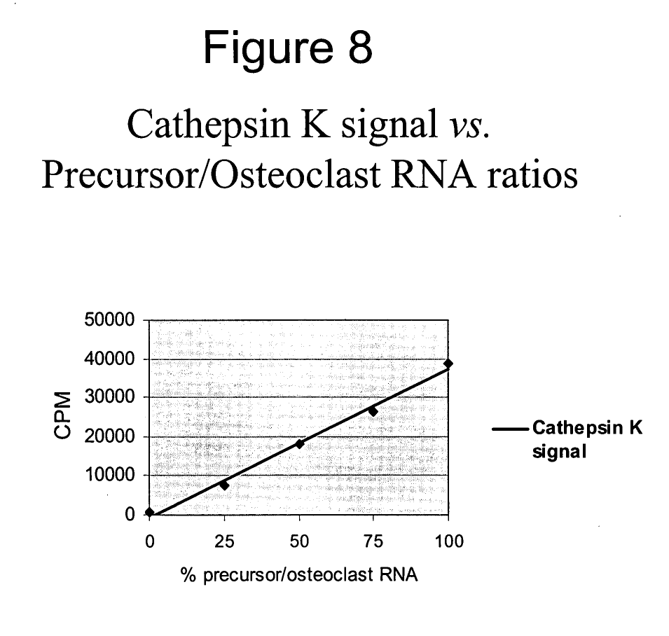

FIG. 7 at A, Lanes 1-5 show the library of linearly transcribed RNA synthesized from the second DNA templates corresponding to the various RNA and RNA mixtures and in all cases, the profile of the transcribed RNA appear to be similar. FIG. 7 at B, Lanes 1-5 show the Northern blot hybridization results for the cathepsin K gene—Lane 1, representing the 100% precursor RNA, showed no cathepsin K signal since this is an osteoclast-specific gene and is not expected to be seen in the precursor sample. However, Lanes 2-5 show increasing levels of the cathepsin K gene corresponding to the increasing starting amounts of osteoclast RNA (25%-100%) in each RNA mixture. In order to quantify the cathepsin K signal, each of the five lanes of the Northern blot was excised and the radioactivity measured by scintillation counting. The counts per minute (cpm) obtained for each of the five lanes, minus the background, was then plotted against the corresponding total RNA or RNA mixtures. As shown in FIG. 8, a linear relationship between the increasing levels of osteoclast total RNA in the RNA mixture and the level of cathepsin K signal was observed. This indicates that the tagging procedure does not appear to introduce a bias for this targeted sequence within the total RNA input range tested.

FIG. 7 contains the following:
Lane 1—500 ng transcribed RNA from 100% precursor
Lane 2—500 ng transcribed RNA from 75% precursor+25% osteoclast
Lane 3—500 ng transcribed RNA from 50% precursor+50% osteoclast
Lane 4—500 ng transcribed RNA from 25% precursor+75% osteoclast
Lane 5—500 ng transcribed RNA from 100% osteoclast

### EXAMPLE 6

### Sensitivity of the Selective Terminal Tagging Procedure

Total RNA was extracted from aliquots of 1000, 5000, 10000, 50000, 100000 and 1000000 undifferentiated mouse RAW 264.7 cells by a Trizol method (Invitrogen) and purified further by RNeasy (Qiagen). The 1 million RAW264.7 cells sample yielded 27.4 μg of total RNA, of which approximately 1% (270 ng) was mRNA. The amounts of total RNA purified from the 1000-100000 samples were not quantified. Rather, the whole amount of total RNA extracted from each cell dilution was used directly in the tagging and transcription procedures. In addition, dilutions of total RNA isolated from the 1 million cells sample representative of 1000, 5000, 10000, 50000 and 100000 cells were similarly tagged and transcribed, in order to determine the efficiency of the method. The mRNA population in each RNA sample was used for making first-strand cDNA and each cDNA was tagged with the oligonucleotide sequence tag template (Seq. ID. No. 2) to generate first DNA templates and purified according to Example 1. Each first DNA templates was subsequently annealed to the second oligonucleotide containing a sequence tag complement to the tag contained in the first DNA templates and a T7 promoter sequence (Seq. ID. No. 3), and an enzymatic DNA polymerization reaction for each performed as described in Example 2. The resulting second DNA templates containing the double-stranded T7 promoter for each reaction was purified and transcribed in vitro using T7 RNA polymerase as described in Example 2. In order to perform a second round of transcription, the transcribed RNA produced from the first transcription reaction for each sample was used to synthesize first-strand cDNA according to Example 1. Each cDNA mixture was then used with the second oligonucleotide template (Seq. ID. No. 3) for second-strand DNA synthesis. Then, each resulting double-stranded T7 promoter containing DNA templates was transcribed using T7 RNA polymerase, according to Example 2. The quantity of RNA obtained for each total RNA sample after two rounds of transcription is summarized in Table 2. Table 2 shows the sensitivity of the terminal tagging procedure comparing purified total RNA diluted from a concentrated stock or purified directly from dilutions of cells.

TABLE 2

| | |
|---|---|
| Total RNA | Amplified RNA (μg) |

| Cell Number | (ng)* | RNA Dilution | Cell |
|---|---|---|---|
| 100000 | 2700 | 73 | 30 |
| 50000 | 1400 | 41 | 9.6 |
| 10000 | 270 | 9.6 | 2.4 |
| 5000 | 140 | 5.9 | 2.2 |
| 1000 | 27 | 1.2 | — |

*based on recovery of 27 μg total RNA from 10⁶ cells

Although the quantity of amplified RNA was linear with respect to the amount of input RNA, the relative amplification efficiency increase throughout the range. An aliquot of 27 ng of total RNA, representing 270 pg of mRNA and 1000 cells, produced 1.2 μg of amplified RNA, an amplification efficiency of 4400 fold. However, no amplified RNA was detected using RNA that was extracted directly from 1000 cells. This is likely due to non-quantitative recovery of RNA from the small sample. With the existing methods of RNA extraction, 5000 cells are involved for the direct amplification of RNA. In an average of 2 experiments, 2.2 μg RNA were amplified from the total RNA that was extracted directly from 5000 cells. By improving the recovery of RNA from small samples, we can expect at least 1 μg of amplified RNA from 1000-2000 cells.

1. A method for adding a terminal sequence tag to nucleic acid molecules, the method comprising: contacting the nucleic acid molecules with a mixture of oligonucleotides, each oligonucleotide having a sequence tag template, a random sequence and a blocked 3′ terminus, under conditions in which the random sequence anneals with the nucleic acid molecules and the nucleic acid molecules are extended to form first DNA or RNA templates using the sequence tag template as template.

2. The method according to claim 1, wherein the nucleic acid molecules comprise DNA.

3. The method according to claim 2, wherein the DNA is complementary DNA.

4. The method according to claim 2, wherein the DNA is formed by strand separation of double-stranded DNA fragments.

5. The method according to claim 1, wherein each oligonucleotide further comprises a promoter sequence, such that the first DNA templates include a double-stranded promoter sequence.

6. A method for synthesizing RNA from DNA templates, the method comprising synthesizing RNA from a complete or partial double-stranded first DNA template including a double-stranded promoter, the complete or partial double-stranded first DNA template being obtained according to the method of claim 5.

7. The method according to claim 1, wherein the nucleic acid molecules are RNA.

8. The method according to claim 7, wherein the RNA is mRNA.

9. The method according to claim 1, further comprising annealing first RNA templates with a second oligonucleotide having a sequence tag complement to the sequence tag contained in the first RNA templates and extending the first RNA templates using the second oligonucleotide as template to form second RNA templates.

10. The method according to claim 9, wherein the second oligonucleotide contains a promoter sequence.

11. The method according to claim 9, wherein the second oligonucleotide comprises at least one restriction endonuclease site.

12. The method according to claim 9, wherein the second oligonucleotide is capable of serving as a primer for primer extension in an enzymatic DNA polymerization reaction using the first RNA templates as template to form RNA:DNA hybrids, wherein DNA strands of the RNA:DNA hybrids are complementary second DNA templates.

13. The method according to claim 12, wherein the complementary second DNA templates are randomly primed in an enzymatic DNA polymerization reaction to form double-stranded DNA templates containing a 3′ double-stranded promoter sequence.

14. A method for synthesizing antisense RNA from DNA templates, the method comprising synthesizing antisense RNA from double-stranded DNA templates including a 3′ double-stranded promoter sequence obtained according to the method of claim 13.

15. The method according to claim 9, wherein the second oligonucleotide contains a blocked 3′ terminus.

16. The method according to claim 1, further comprising annealing first DNA templates with a second oligonucleotide having a sequence tag complement to the sequence tag contained in the first DNA templates, and the first DNA templates are extended using the second oligonucleotide as template to form second DNA templates.

17. The method according to claim 16, wherein the second oligonucleotide contains a promoter sequence.

18. The method according to claim 16, wherein the second oligonucleotide further comprises at least one restriction endonuclease site.

19. The method according to claim 16, wherein the second oligonucleotide contains a blocked 3′ terminus.

20. A method for synthesizing RNA from DNA templates, the method comprising synthesizing RNA from a complete or partial double-stranded second DNA template including a double-stranded promoter, the complete or partial double-stranded second DNA template being obtained according to the method of claim 16.

21. A method for adding a terminal sequence tag to the 3′-end of a first nucleic acid molecule, the method comprising: contacting the first nucleic acid molecule with a mixture of oligonucleotides, each oligonucleotide having a sequence tag template, and a blocked random sequence at its 3′ terminus, under conditions in which the random sequence of at least one of the oligonucleotides anneals with the first nucleic acid molecule and the first nucleic acid molecule is extended to form a first extended nucleic acid molecule using the sequence tag template as template.

22. The method of claim 21, further comprising: separating the annealed first extended nucleic acid molecule and the oligonucleotide from each other and contacting the first extended nucleic acid molecule with an oligonucleotide having at least a portion of said sequence tag template and being unblocked at its 3′-terminus under conditions in which the at least a portion of said tag template anneals to its complement in the first extended nucleic acid molecule and the 3′-end of the unblocked oligonucleotide is extended to form a second extended nucleic acid molecule using the first extended nucleic acid molecule as template.

23. The method of claim 22, wherein the sequence tag template unblocked at its 3′-terminus further comprises a promoter sequence at its 5′-end, and the first extended nucleic acid molecule is extended at its 3′-end using the promoter sequence as template.

24. The method of claim 23, wherein the first nucleic acid molecule is DNA.

25. A method for synthesizing sense RNA from DNA molecules comprising forming first DNA templates by contacting the DNA molecules with a mixture of oligonucleotides, each having a sequence tag template, a random sequence and a blocked 3′ terminus, under conditions such that, the DNA molecules anneal with the random sequence and are extended using the sequence tag template as template;
forming first DNA templates having a 5′ double-stranded promoter sequence by contacting the DNA molecules with a mixture of oligonucleotides, each having a promoter sequence, a sequence tag template, a random sequence and a blocked 3′ terminus, under conditions such that, the DNA molecules anneal with the random sequence and are extended using the promoter sequence and the sequence tag templates as template;
forming second DNA templates having a 5′ double-stranded promoter sequence by contacting the first DNA templates with a second oligonucleotide, containing a sequence tag complement to the sequence tag contained in the first DNA templates and a promoter sequence, under conditions such that, the first DNA templates anneal with the second oligonucleotide and are extended using the promoter sequence as template;
forming second DNA templates having a 5′ double-stranded promoter sequence by contacting first DNA templates with a second oligonucleotide, containing a sequence tag complement to the sequence tag contained in the first DNA templates, a promoter sequence and a blocked 3′ terminus, under conditions such that, the first DNA templates anneal with the second oligonucleotide and are extended using the promoter sequence as template; and
synthesizing RNA from the first or second DNA templates having a 5′ double-stranded promoter sequence.

26. The method according to claim 25, wherein the DNA molecules are formed by contacting a mixture containing mRNA with a primer having a terminal sequence complementary to the mRNA, under conditions such that, the terminal sequence of the primer anneals with the mRNA and is extended using the mRNA as template.

27. The method according to claim 25, wherein the method further comprises extending the second oligonucleotide using the first DNA templates as template to form complete double-stranded second DNA templates

28. A method for amplifying terminal sequences of DNA molecules comprising,
forming first DNA templates by contacting the DNA molecules with a mixture of oligonucleotides, each having a sequence tag template, a random sequence and a blocked 3′ terminus, under conditions such that, the DNA templates anneal with the random sequence and are extended using the sequence tag template as template;
forming double-stranded DNA templates by contacting the first DNA templates with a first primer having a sequence complementary to the sequence tag contained in the first DNA templates, under conditions such that, the sequence of the first primer anneals with the sequence tag contained in the first DNA templates and is extended using the first DNA templates as template to form double-stranded DNA templates;
amplifying the double-stranded DNA templates by contacting with said first primer and a second primer containing a sequence complementary to a sequence from the complementary strand of the first DNA templates, under conditions such that the primers anneal to complementary templates and are extended repeatedly; and
forming complementary second DNA templates according to claim 12 and amplifying said complementary second DNA templates by contacting with a second primer containing a sequence that is complementary to the complementary second DNA templates and a first primer having a sequence of similar sense to the sequence tag contained in the complementary second DNA templates, under conditions such that the primers anneal to complementary templates and are extended repeatedly.

29. The method according to claim 28, wherein the DNA molecules are formed by contacting a mixture containing mRNA with a primer having a sequence complementary to the mRNA, under conditions such that, the sequence of the primer anneals with the mRNA and is extended using the mRNA as template.

30. The method according to claim 28, wherein the second primer further comprises at least one sequence that is specific for at least one sequence in a mixture of double-stranded DNA templates, wherein a limited number of sequences in the mixture are amplified.

31. A composition comprising a nucleic acid molecule having annealed at its 3′-end an oligonucleotide blocked from replication at its 3′-end and a sequence tag template at its 5′-end, the sequence tag template extending past the 3′-end of the nucleic acid molecule.

32. The composition of claim 31, wherein the nucleic acid molecule is DNA.

33. The composition of claim 31, wherein the nucleic acid molecule is RNA.

34. The composition of claim 31, wherein the oligonucleotide further comprises a promoter sequence.

35. The composition of claim 31, wherein the nucleic acid molecule further comprises a double-stranded promoter sequence.

36. A kit for selective terminal tagging of nucleic acid molecules in a sample, the kit comprising: (a) an oligonucleotide sequence tag template further comprising a tag sequence, and a random sequence for binding terminally said nucleic acid molecules, wherein the 3′-end of said oligonucleotide sequence tag template is blocked to preclude extension thereof; (b) a DNA polymerase and reagents for extending the 3′ terminal ends of said nucleic acid molecules; (c) size selection columns and buffers for removal of unused oligonucleotide sequence tag template; and (d) instructions for hybridizing said oligonucleotide sequence tag template to nucleic molecules of said sample, extending said nucleic acid molecules with the DNA polymerase using the oligonucleotide sequence tag templates as template, and creating first DNA or RNA templates with double-stranded regions corresponding in sequence to at least a sequence tag region.

37. The kit of claim 36, further comprising a second oligonucleotide sequence tag template having a sequence tag complement to the oligonucleotide sequence tag contained in the first RNA or DNA templates and a promoter sequence, wherein the first RNA or DNA templates are extended according to instructions included using the second oligonucleotide sequence tag template as template to form second DNA templates or second RNA templates containing a complementary promoter sequence.

38. The kit of claim 37, wherein the second oligonucleotide sequence tag template contains a blocked 3′ terminus.

39. The kit of claim 36, wherein the oligonucleotide sequence tag template further comprises a promoter sequence on it's 5′-end.

40. The kit of claim 36, further comprising a reverse transcriptase, at least one enzyme for RNA hydrolysis and an oligonucleotide complementary to mRNA molecules.

41. The kit of claim 40, further comprising a RNA polymerase matching a functional promoter and reagents, and cofactors for in vitro RNA synthesis from said promoter.

42. The kit of claim 40, further comprising a first primer corresponding to the sequence tag and at least one second primer for exponential amplification of at least one sequence contained in nucleic acid mixture of said sample, wherein the primers anneal to complementary templates and are extended repeatedly.
